(19) United States
(12) Patent Application Publication
(10) Pub. No.: US 2016/0007913 A1
(43) Pub. Date: Jan. 14, 2016

(54) SYSTEM FOR MONITORING A STATE OF DISEASE

(71) Applicant: Lone DARKET, Kgs. Lyngby (DK)

(72) Inventor: Lone DARKET, Kgs. Lyngby (DK)

(21) Appl. No.: 14/765,705

(22) PCT Filed: Feb. 4, 2014
(86) PCT No.: PCT/EP2014/052159

§ 371 Date: Aug. 4, 2015

(30) Foreign Application Priority Data
Feb. 13, 2013 (EP) 13155060.0

(51) Int. Cl. A61B 5/00; A61B 5/087

(52) U.S. Cl. CPC A61B 5/486; A61B 5/087; A61B 5/4842; A61B 5/7246; A61B 5/0022; A61B 5/7475; A61B 5/7275; A61B 2560/0475; A61M 2205/52

(57) ABSTRACT

The present invention relates to a device, a system and a method for measuring the lung function and the inhalation technique of a human subject, to facilitate the empowerment of patients to self-manage airway diseases, in particular asthma. The device according to the invention comprise a first part capable of measuring the time (s) and volume of air/time (v/s) exhaled from the human subject during a single inhalation, thereby providing a measurement of the lung function, and further comprising a second part capable of measuring the acceleration (m/s²) of air and volume of inhalation from the human subject during a single inhalation, thereby providing a measurement of the inhalation technique, the second part of the device further comprising means for applying metered resistance to the inhalation, the applied resistance impairing the acceleration (m/s2) of air and volume of inhalation from the human subject during the inhalation.

### TECHNICAL FIELD

The present invention relates to methods and systems for monitoring the state of disease in the airways of a human or animal subject.

### BACKGROUND

Asthma is a chronic inflammatory disorder of the airways with airway hyperresponsiveness that leads to recurrent episodes of wheezing, breathlessness, chest tightness and coughing, particularly at night or in the early morning. These episodes are usually associated with widespread but variable airflow obstruction within the lung that is reversible either spontaneously or with treatment. The symptoms of asthma are usually reversible and significantly affected by environmental conditions surrounding the patient at any point in time and e.g. affected by random infections such as cold and flue.

Although, there are currently no precise physiologic, immunologic, or histologic tests for diagnosing asthma, there is broad consensus that physicians should use spirometry whenever possible to guide the diagnosis and management of asthma. The diagnosis is made based on the pattern of symptoms (airways obstruction and hyperresponsiveness) and the response to therapy (partial or complete reversibility) over time. Asthma is confirmed when a patient responds to asthma treatment, thereby confirming the reversibility of airway obstruction after treatment. The frequency of the disease has increased significantly since the 1970s. In 2010, 300 million people were affected worldwide, and in 2009 asthma was estimated to be responsible for 250,000 deaths on a global scale. Asthma affects approximately 7% of the population of the United States and 5% of the population in the United Kingdom.

The development of asthma and the severity of the disease are highly dependent on environmental and behavioural factors. These factors significantly influence how severe asthma is and how well it responds to different types and different doses of medication. Many environmental risk factors have been associated with asthma, such as exposure to indoor and outdoor air pollutants (e.g. household chemicals such as phthalates in PVC and perfume, pet allergens, dust mite allergens etc. traffic, animals and outdoor chemicals), tobacco smoking, high ozone levels and particularly endotoxin exposure.

Use of medicine for treatment of certain conditions (e.g. the use of beta blocker medications such as metoprolol) may trigger asthma. Further bacterial and viral respiratory infections have an effect on asthma symptoms, and may trigger exacerbations. Even further, environmental conditions such as temperature, air pressure and humidity may play a significant role in the actual state of disease in a subject suffering from asthma.

Even further, behavioural induced conditions (e.g. sports, travel and transportation) and even psychological stress may trigger symptoms of asthma.

Asthma also occur as a result of (or is worsened by) exposure to triggering agents in the surroundings of the individual suffering from asthma, e.g. in the work place. A significant amount of individuals suffering from asthma induced by conditions at work are not reported or are not recognised as such. When recognised, these work-related triggering agents can be dealt with, thereby reducing the risk of disease.

Subjects suffering from asthma may be stable (free or substantially free of symptoms) for weeks or months and then suddenly develop an episode of acute asthma. An acute asthma exacerbation is commonly referred to as an asthma attack. An asthma attack is when symptoms are worse than usual. They can come suddenly and can be mild, moderate or severe. The classic symptoms are shortness of breath, wheezing, and chest tightness. In a mild exacerbation, the peak expiratory flow rate (PEFR) is ≧200 L/min or ≧50% of the predicted best. Moderate is defined as between 80 and 200 L/min or 25% and 50% of the predicted best, while severe is defined as ≦80 L/min or ≦5.25% of the predicted best. However, there may be substantial variations in PEFR between individuals, e.g. children, men and women, age, height and weight. Often the event triggering sudden exacerbations are unknown to the subject suffering from the disease, and further, different patients react differently to various factors. Many patients may develop severe exacerbation of asthma from several different (known or unknown) triggering agents (e.g. agent as described above).

Asthma may also be exercise-induced, and a diagnosis of asthma is common among top athletes. There appears to be a relatively high incidence of asthma in sports such as cycling, mountain biking, swimming and long-distance running, and a relatively lower incidence in weightlifting and diving.

Medicinal practitioners may have difficulties in determining the actual state of disease, the severity of the disease and the events triggering the disease as the subjects suffering from asthma may e.g. be free of symptoms when consulting the medicinal practitioner. Further, the medicinal practitioner may fail to reliably determine or examine variations in the interim between visits.

Thus, determining the correct treatment regimen, the optimal behaviour to prevent asthma attacks and the correct way of taking the medicaments cannot be adequately performed by the medicinal practitioner. Thus, asthma is an incurable and highly variable disease that, for optimal treatment, requires highly developed disease-managing skills by the patient suffering from the disease.

There is no cure for asthma. However, symptoms can typically be improved, even to a stage where the subject suffering from the disease has no real symptoms. The more specific and customised the plan for proactively monitoring and managing the symptoms, the less affected by the disease the subject will be. Correctly treated, controlled and with adequate management, a person with asthma can live a normal and active life. The most effective treatment for asthma is identifying triggers and—if possible—eliminating or at least minimizing exposure to them. If trigger avoidance is insufficient, medication is needed.

Pharmaceutical drugs for treatment of asthma are selected based on e.g. the severity of illness and the frequency of symptoms. Bronchodilators are recommended for short-term relief of symptoms. Short-acting beta2-adrenoceptor agonists (SABA), such as salbutamol, are the first line treatment for asthma symptoms. In patients suffering from only occasional attacks, no other medication is needed. Anticholinergic medications, such as ipratropium bromide, provide additional benefit when used in combination with SABA in those with moderate or severe symptoms. Anticholinergic bronchodilators can also be used if a person cannot tolerate SABA. In case of a mild, persistent disease (more than two attacks a week), low-dose inhaled glucocorticoids or alternatively, an oral leukotriene antagonist or a mast cell stabilizer is recommended. For those who have daily attacks, a higher dose of inhaled glucocorticoid is used. In a severe asthma exacerbation, oral glucocorticoids are added to these treatments. Glucocorticoids are generally considered the most effective treatment available for long term control. Inhaled forms are usually used except in the case of severe persistent disease, in which oral steroids may be needed.

It is usually recommended that inhaled formulations be used once or twice daily, depending on the severity of symptoms. Long acting beta-adrenoceptor agonists (LABA) have at least a 12-hour effect. However, they should not be used without an accompanying steroid due to an increased risk of severe symptoms, including exacerbation of asthma in both children and adults.

Medications are typically delivered using metered-dose inhalers (MDIs) alone or in combination with an asthma spacer or using a dry powder inhaler. The spacer is a plastic cylinder that mixes the medication with air, making it easier to receive a full dose of the drug. A nebulizer may also be used.

The medicaments typically act as topical medicaments on the site of the lungs where the medicament is delivered. Only rarely does the medicament have any significant effect when delivered systemically. When asthma medicaments are inhaled by the patient, the inhalation technique of the subject determines how the medicament is distributed. If the medicament reaches the site of asthma-induced inflammation, the medicament will excerpt its effect topically, whereas if the medicament does not reach the site of asthma-induced inflammation, the medicament will only excerpt a very minor effect systemically.

Chronic obstructive pulmonary disease (COPD) may coexist with asthma and may occur as a complication of chronic asthma. COPD closely resembles asthma in symptoms. After the age of 65, most people with obstructive airway disease will have asthma and COPD. COPD can normally be differentiated by increased airway neutrophils, abnormally increased wall thickness, and increased smooth muscle in the bronchi. COPD and asthma (and other airway diseases) share similar principles of management using the same types of medicaments, i.e. corticosteroids and long acting beta agonists. A range of other diseases in the airways of human or animal subjects are severe and may be life-threatening and, like COPD and asthma, be significantly ameliorated by the right treatment and by monitoring the disease.

In summary, asthma and other diseases in the airways of humans are highly dependent on external environmental conditions, highly dependent on behavioural conditions, and symptoms can be very effectively treated by applying the correct medical treatment.

Correct medical treatment is, however, highly dependent on a very accurate knowledge of the actual state of disease and the inhalation performance (inhalation technique) of the patient during the administration of the drug.

Optimal drug delivery (and optimal dosing) depends of the particular drug (i.e. size distribution), the inhalation device used and the inhalation technique used by the patient. The optimal inhalation technique can be estimated by medicinal practitioners with special training within the field, but ultimately, it is best obtained by training the patient in an individual setting.

Training in inhalation techniques is essential for effectively securing medication to reach and maximize deposition in the airways, and studies have shown that the inhalation technique is generally ineffective or incorrectly performed by the majority of patients (up to 80%).

Training inhalation technique requires knowledge of the state of the disease as it develops in the patient in a real life setting. Thus, to increase patient adherence, the patients need easy access to monitoring the specific particulars of their disease.

Treatment of chronic or semi-chronic airway conditions, such as asthma and COPD, is largely based on patient self-management (usually requiring complex multi-therapies), wherein the daily use of different medical delivery devices and different medicaments and even different brands of the same medicaments for treatment of periodic changes in the condition, and wherein changes in the patient's behaviour, lifestyle and environment affect the correct choice of treatment.

Thus, the patients daily self-administer medicaments and daily face several potentially life-threatening risks. It is generally accepted that increasing the effectiveness of prescribed treatments through improved patient adherence may have a far greater impact on peoples' health than any improvement of specific medical treatments. Studies consistently find significant cost savings and increase the effectiveness of health interventions that are attributable to low-cost interventions for improving adherence. Without a system that addresses the determinants of adherence, the advances in biomedical technology will fail to realise its potential to reduce the burden of chronic illnesses. Access to medication is necessary but insufficient in itself for the successful treatment of disease.

Other airway diseases are similar to asthma in that the diseases and symptoms are best managed by the patient.

The optimal use of appropriately prescribed medicines is vital to the self-management of most chronic illness. Reviews conducted across disease states and countries are consistent in estimating that between 30 and 50 percent of prescribed medication is not taken as recommended. It represents a failure to translate the technological benefits of new medicines into health gains. It is generally known that increasing the effectiveness of adherence interventions may have a far greater impact on the health of the population than any improvement in specific medical treatments (see Haynes, R; McDonald, H; Garg, A and Montague, P (2002)—Interventions for helping patients to follow prescriptions for medications, The Cochrane Database of Systemic Reviews 2).

Further, patient self-management of disease requires adequate skills and management tools. Especially in several research studies it has been demonstrated that only 5-15% of asthma patients are well controlled, even on Glucocorticoids (see e.g. Asthma Insights and Reality in Europe (AIRE), International Asthma Patient Insight Research Study (INSPIRE) and European Community Respiratory Health Survey (ECRHSII)). In three epidemiologic studies almost 7000 patients were included. These studies also documented that approximately 50% of asthma patients are uncontrolled even on Glucocorticoids.

Appropriate self-management of airway diseases, in particular asthma, is further complicated by the fact that the actual state of disease has a great impact on the optimal inhalation technique. Thus the inhalation technique that facilitates the optimal acceleration and flow of inhaled medicaments vary with changes in disease condition. Further, different types of medication require different inhalation techniques. Accordingly, the optimal inhalation technique for one medicament, i.e. a particulate medicament, requires a forceful acceleration in the start of the inhalation, whereas other medicaments, i.e. medicaments delivered as pMDI (pressurized metered dosed inhalers) does not require forceful acceleration, but requires coordination skills of the patient such that the inhalation and the release of the medicament is carefully coordinated by the patient. Further the inspiratory flow rate throughout the inhalation is important. Other important parameters may be the inhalation time within the target flow range and the breath hold time at the end of inhalation.

Accordingly, patient adherence is a major concern for patients suffering from chronic or semi-chronic airway diseases. Accurate assessment of disease states and adherence and adjusting the general behaviour of the patient are necessary measures for the effective and efficient treatment of the disease.

The overall object of the invention was to provide a method, a device and a system enabling better patient adherence and self-management for patients suffering from chronic or semi-chronic airway diseases.

Specifically, there is a need in the art for devices and methods empowering patients suffering from airway diseases to manage their disease on a day-to-day basis. There is a need in the art for empowering patients to identify the disease and the status of the disease, a need in the art for empowering patients to identify triggering events and triggering agents, and a need in the art for empowering patients to train the correct inhalation technique. Further, there is a need in the art for means for collecting individual data on a day-to-day basis to improve the empiric material on which decisions regarding treatment and medicine regime are based.

Often, for example in case of children suffering from airway diseases, the monitoring of the disease and the concurrent monitoring of external factors influencing the disease may prove problematic due to lack of sufficient skills and awareness of handling recordings. Thus, there is a need in the art for systems, devices and methods for gathering and storing information on the state of the disease and combining this information with information on external environmental conditions surrounding the patient.

In particular, there is a need in the art for devices, systems and methods empowering patients suffering from airway diseases, in particular asthma, to monitor the disease continuously. In particular, there is a need in the art for systems, apparatuses and methods empowering patients suffering from airway diseases, in particular asthma, to monitor the disease continuously and to monitor the actual performance of the individual suffering from the disease when taking the required medication.

There is a need in the art for systems, devices and methods that are capable of providing feedback to the patient, e.g. on how to improve the inhalation technique.

There is a need in the art for systems, devices and methods empowering patients suffering from airway diseases, in particular asthma, to identify environmental or behavioural situations triggering the onset of the symptoms of the disease.

There is a need in the art for systems, devices and methods empowering patients suffering from airway diseases, in particular asthma, to improve implementation of health and treatment guidelines, and a need in the art for systems, devices and methods which are easily operated to support easy monitoring of the disease, and easy (automated) storage of relevant data.

Accordingly, it is an object of the invention to empower patients suffering from airway diseases to correctly control their disease in every situation.

It is an object of the invention to provide devices, systems and methods empowering patients suffering from airway diseases to continuously monitor the state of their disease. Specifically, there is a need for a monitoring device, system and methods providing feedback to the patient for monitoring the disease. Even more specifically, there is a need for a monitoring device, system and methods providing feedback to guide the patient in the monitoring and managing of the disease.

It is an object of the invention to provide devices, systems and methods empowering patients suffering from airway diseases to improve the efficacy of their inhalation techniques.

It is an object of the invention to provide devices, systems and methods empowering patients suffering from airway diseases to improve their treatment of the disease.

It is an object of the invention to provide devices, systems and methods empowering patients suffering from airway diseases to continuously improve their treatment of the disease in response to rapid changes of external conditions affecting the state of disease.

It is an object of the invention to provide devices, systems and methods empowering patients suffering from airway diseases to continuously monitor the state of their disease in response to rapid changes of the disease conditions.

It is an object of the invention to provide devices, systems and methods empowering patients suffering from airway diseases to identify allergens or environmental conditions triggering the onset of the disease and to adapt the behaviour of the patients so as to avoid allergens and situations triggering the disease.

It was further an object of the invention to provide patients, caregivers & healthcare providers with improved systems and devices in order to better manage the general treatment of airway diseases such as asthma and COPD. A further object was ensuring that patients continue therapy for their chronic condition for long periods of time.

It was a further object to provide the patients with the opportunity to improve the daily life and to minimise the impact of the disease, through 1) optimised intake of prescribed medication through training of inhalation techniques and 2) through better understanding of the relationship between the individual disease condition and the environment in which the individual lives and acts. It was another object of the invention to provide the patients with a personal perception of the relationship between the disease and effectiveness of inhaled treatment/medication, thereby providing the patient with the opportunity to learn to manage the disease without the aid of the system according to the invention.

Systems and devices for continuously monitoring general lung functions of patients suffering from asthma are known. SpiroSmart is a mobile phone-based platform that allows for the analysis of common lung function measures (FEV1, FVC, PEF). By analysing lip reverberation, these systems are capable of monitoring pulmonary ailments such as asthma, chronic obstructive pulmonary disease, and cystic fibrosis. The SpiroSmart system is, however, not able to monitor the inhalation technique of the patient suffering from the disease, and is not able to provide feed-back empowering the patient to learn about his condition and to improve the inhalation techniques in response to particular environmental events.

Furthermore, devices capable of assessing the inhalation technique of patients are known. One example is the Vitalograph AIM™.

U.S. Pat. No. 6,015,388A discloses a method where an estimate of the peak inspiratory flow and/or the peak inspiratory acceleration is obtained indirectly by continuous monitoring of a breath waveform (obtained by measuring the rib cage motion and the abdominal motion of the subject). This method is suited as an alternative to direct measurements of peak inspiratory flow and/or the peak inspiratory acceleration in certain situations, e.g. in situations where long term monitoring (e.g. during sleep) of respiratory drive is wanted. The system and method according to U.S. Pat. No. 6,015,388A differ fundamentally from the system and method according to the present invention e.g. in that peak inspiratory flow and/or the peak inspiratory acceleration are estimated by using long-term monitoring of breath waveforms.

### SUMMARY OF THE INVENTION

The invention relates to a device and a system providing easy and patient-friendly assessment of their disease and assessment of their technique in taking their medication(s). Further, the inventive system preferably provides automated data processing, whereby the important particulars of the disease and the inhalation technique and/or the “real-life” situation of the user (e.g. GPS position) are stored and processed automatically, thus enabling the patient to learn how to adapt the behaviour and learn which events and situations to avoid.

The method according to the invention also relates to a method of training the individual to optimise the behavioural response to the disease. The method also relates to a method of training the individual to optimise the way to administer doses of a particular medicament.

In one aspect, the invention comprises a medical device or system enabling the individual patient to estimate the Lung Function (Spirometry) and a device capable of estimating the inhalation technique of the patient based on the performance (inhalation technique) of the patient with respect to the patients prescribed inhalers.

Further, in a preferred aspect, the invention comprises providing the means of easy access to a caregiver who is able to monitor the patient's state of disease and who is able to provide immediate support to the patient.

In addition, in a preferred aspect, the invention comprises a device providing the means of easy access to a community capable of providing general support to the patient. Such general support could e.g. comprise access to health records, care plans and treatment regimes including e.g. a prescription overview.

### BRIEF DESCRIPTION OF THE FIGURES

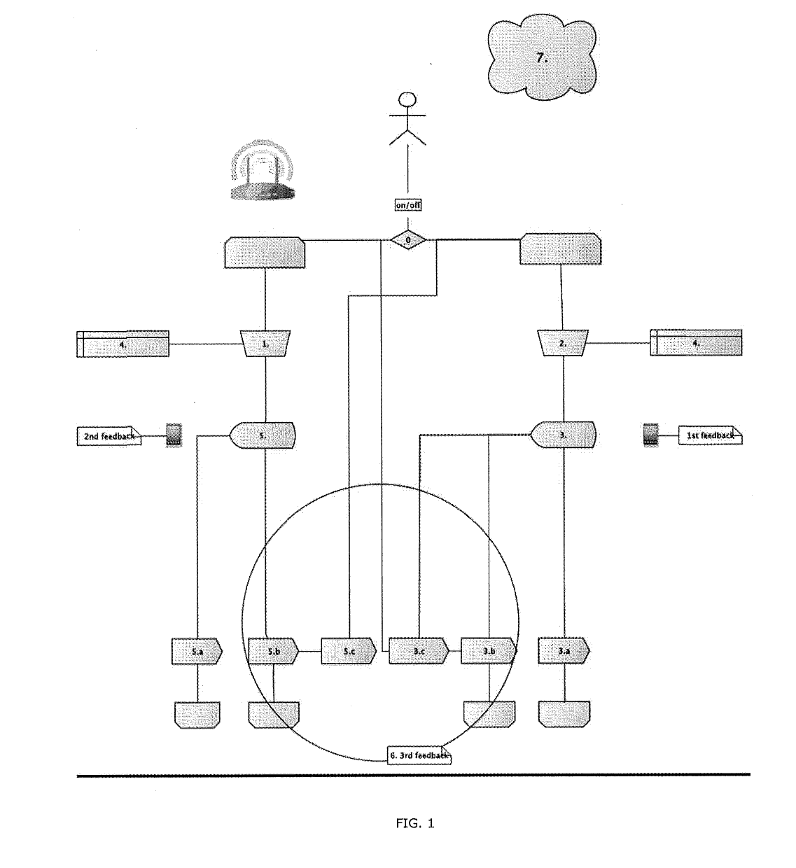

FIG. 1 shows a preferred embodiment of the system according to the present invention;
0. The user selects either trainer (inhale-determining function) or spirometer (exhale-determining function).
1. Select the type of inhaler device/medicament (pre-set resistance applied; resembling the resistance of the selected type of inhaler). Perform inhalation.
2. Perform spirometry (exhale).
3. Display feedback (first feedback recommending action) 3a: lung function OK, 3b. suitable drug substance, 3c. train inhalation technique.
4. Data storage means
5. Display feedback (second feedback recommending action): either 5a: the inhalation technique is OK, or 5b. an alternative inhalation technique is suggested, or 5c. the lung function is to be tested.
6. Display feedback (third feedback recommending action); a suggestion of actions recommending either 3b, 3c, 5b, 5c.
7. WIFI and/or connection to mobile device.

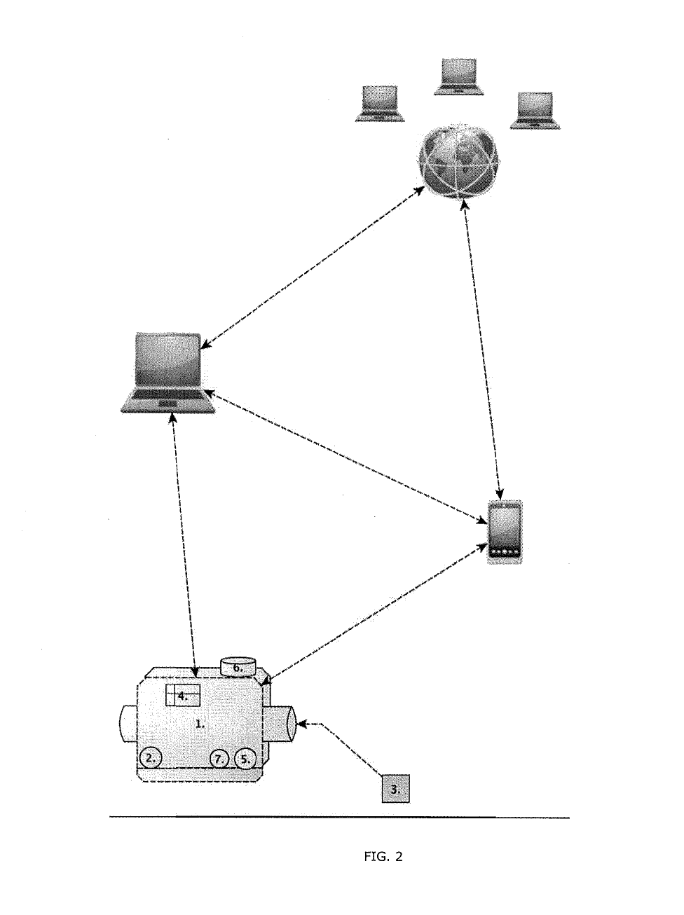

FIG. 2 shows a preferred embodiment of the system according to the present invention;
1. Device
2. On/Off
3. Mouth piece, inhale/exhale
4. Data storage means (controller)
5. Position tracker
6. pMDI dose-release simulator button
7. Means for transmitting data wireless

### DETAILED DESCRIPTION OF THE INVENTION

One essential part in achieving the objects of the invention was the provision of a new portable device capable of 1) measuring the lung function and 2) measuring the inhalation technique with respect to one or more, preferably at least two or more, different conventional inhalation devices (also called inhalers or prescription inhalation devices).

Devices capable of measuring the lung function are widely used and are known as spirometers. The lung function may be estimated by e.g. measuring the time (s) and volume of air/time (v/s) exhaled from the human subject during a single inhalation. The skilled person is familiar with the structure and manufacture of such devices.

Devices capable of measuring the inhalation technique are also known. The inhalation technique with respect to some types of prescribed medication may be assayed by measuring the acceleration (m/s²) of air and time and volume of inhalation from the human subject during a single inhalation. Some medicaments require a forceful acceleration, while others do not benefit from a to forceful acceleration. Inhalation technique with respect to other types of medication may be assayed by measuring the time of activation of the medicament (e.g. pMDI) and the timing of the inhalation, thereby estimating the correct coordination of events when taking the medicament.

Adequate and optimal inhalation techniques vary from device to device and (in case of particulate medicaments) vary with the type of medicament and the particle size of the medicaments. Manufacturers of inhalation devices currently on the market publish the adequate and optimal inhalation technique for each particular medicament. In addition, the skilled person is familiar with the structure and manufacture of inhaler devices.

However, the combination of the two devices with a metered (adjustable) resistance applied to the inhalation monitoring (second) part of the device is, to the best of the knowledge of the inventors, not known. The metered resistance should preferable provide the user with the ability to set the resistance to a value substantially identical to the resistance that is characteristic of the particular inhalation device(s) and medicaments used by the patient. In this way, the patient will be able to monitor the lung function and inhalation technique with respect to one or more inhalers using only one device. Resistance values for specific devices and medicaments are usually published by the inhaler manufacturers. Further the patient will be able to estimate the optimal inhalation technique relative to the state of disease (lung function).

Thus, in a first aspect, the invention is a device for measuring the lung function and the inhalation technique of a human subject, the device comprising a first part capable of measuring the time (s) and volume of air/time (v/s) exhaled from the human subject during a single inhalation, thereby providing a measurement of lung function and a second part capable of measuring the acceleration (m/s2) of air and volume of inhalation from the human subject during a single inhalation, thereby providing a measurement of inhalation technique, where the second part of the device further comprises means for applying metered resistance to the inhalation, where the applied resistance impairs the acceleration (m/s2) of air and volume of inhalation from the human subject during the inhalation.

Preferably, the second part of the device is also capable of measuring the coordination of the patient in triggering the inhalers (releasing the medicament) and performing the inhalation, thereby providing a measurement of inhalation technique with respect to certain types of medicaments (medicaments provided in pressurized metered dosed inhalers).

Preferably, the second part of the device is also capable of measuring the inhalation time within a given target flow range. Preferably, the second part of the device is also capable of measuring the breath hold time at the end of inhalation.

The use of such device results in a tremendous improvement of the everyday life of the patient, an improved adherence and a more efficient use of the particular medicament inhaled by the patient. In turn, this will also lead to fewer side effects due to a more efficient dosage use.

Patients more often than not simultaneously use more than one inhaler. The optimal inhalation technique varies for each type of inhaler, and with each type of medication. The optimal inhalation technique also varies with the particle size of the medicament that is to be taken. The optimal inhalation technique for each inhaler varies from a fast and forceful inhalation (powerful acceleration) to a slow and prolonged inhalation without powerful acceleration. Further, for some medicaments the coordination between inhalation and triggering of the inhalation device (release of medicament) is essential.

Thus, it is preferred that two or more, such as three or more, predetermined resistance values may be applied to the inhalation (second) part of the device. Thereby, the patient is able to monitor the inhalation technique with respect to multiple devices, thus increasing the adherence and technique of the patient and the medicament.

In order to increase patient comfort and adherence, it is preferred that the inhaler part (second part) and the part measuring the lung function (first part) are integrated into a single unit such that the inhale air and exhale air flow through the same path, at least at the entry point of the exhale air and the exit point of the inhaled air. Thereby, the user may exhale and inhale in the device without removing the device from the mouth. Hence, in a preferred aspect, the invention relates to a device wherein the first part and the second part are integrated into a single unit, where the entry point of exhaled air is at the same location as the exit point of inhaled air.

It is further highly preferred that the device is portable. Portable means that it is possible for a normal human subject to carry it around in everyday life situations. Preferably, the device weighs less than 2 kg, such as less than 1 kg, such as less than 500 g.

In order to substantially improve the everyday life of patients suffering from airway diseases, it is highly preferred that the device is integrated in a system that comprises means that may uptake and store lung function or other data supplied by the device. Preferably, the data uptake and storage are performed automatically and electronically. The means for taking and storing data are preferably electronic means, may be selected among minicomputers, mobile phone apps and may include systems that take up data and store the data in the device (e.g. a microcontroller) and/or transmit data for electronic storage elsewhere (e.g. using blue tooth). The collected and stored data may be transferred subsequently when connected to wifi or connected to another mobile data unit.

It is highly preferred that the means for data storage is capable of comparing the actual measurement with standard data for the patient (lung function data representative of the state where the patient is optimally treated and essentially free of disease symptoms), thereby providing a measure of the relative lung function. The relative lung function is directly related to the state of disease in the patient, and the system will thereby be able to provide the patient with valuable insight as to his/her condition at any time. This will increase adherence and will also enable and empower the patient to respond faster and more adequately to changes in his/her condition.

These changes in condition may arrive suddenly without notice due to unforeseeable factors affecting the disease. Further, changes in condition may be observed to fluctuate in daily, weekly, and even monthly patterns. The system according to the invention will therefore be able to empower the patient to identify conditions triggering the disease as well as disease patterns, thereby providing the patient with an opportunity to respond adequately to reoccurring symptoms. Further, changes in condition may be foreseen in response to changing environments, such as e.g. travel, new situations, sports as well as infectious diseases and stress.

In time, the patient may even learn so much from the system that the patient will be able to monitor the disease without the system.

Thus, in another highly preferred aspect, the invention relates to a system for monitoring the state of disease in the airways of a human or animal subject, said system comprising a device as described above, a data storage device comprising means for collecting and storing one or more measurements of the lung function, and means for comparing a measurement of the lung function with previously obtained measurements of the lung function from the human or animal subject, thus obtaining a measurement of relative lung function.

Further, it is highly preferred that the system comprises means for providing appropriate responses from the system to the subject, based on the measured lung function and/or inhalation technique.

An appropriate response to the measurement of the relative lung function would be to suggest the patient to take a particular medicament in response to one relative lung function, and another particular medicament in response to another relative lung function. The adequate response should preferable be determined by a trained doctor by assigning to the system values of relative lung function at which each medicament should be administered. The adequate response can be integrated in the device or system e.g. by using prescribed care plans, medicine regimes or treatment algorithms.

Thus, in another highly preferred aspect, the invention relates to a system as described above comprising means for providing a first feedback to the human or animal subject, the first feedback being to suggest a suitable drug substance for treating the human or animal subject, the suggestion being based on a particular measurement of the relative lung function.

In order to substantially improve the everyday life of patients suffering from airway diseases, it is further highly preferred that the device is integrated in a system that comprises means to uptake and store data about the inhalation technique supplied by the device. Preferably, the data uptake and storage are performed automatically and electronically. The means for taking and storing data are preferably electronic means, may be selected among minicomputers, mobile phone apps and may include systems that take up data and transmit data for electronic storage elsewhere (e.g. using blue tooth).

It is highly preferred that the means for data storage is capable of comparing the actual measurement of inhalation technique with standard data of either optimal or sufficient inhalation for the particular inhaler and medicament that the patient uses, thereby providing a measure of the relative inhalation technique.

The relative inhalation technique is directly related to the amount of the prescribed medicament reaching the desired place of action in the airways of the patient. This aspect of the invention will provide the patient with valuable insight regarding the efficacy of the medicament at the particular inhalation technique, and will provide the patient with information if the inhalation technique is inadequate or sub-optimal.

The measurement of (relative) inhalation technique preferably comprises data regarding the timing of inhalation in relation to triggering release of a medicament, whereby the user may determine and train the coordination of inhalation and triggering the release of the medicament.

This data may be generated by pressing a dose-release simulator device and determining the timing of the release of medicament compared to the timing of the inhalation.

Thus, it is preferred that the system according to the invention comprises means for comparing the timing of the release of medicament and the timing of the initiation of inhalation.

As the inhalation technique of the patient (the acts required to obtain a particular acceleration and inhalation volume, as well as coordination of inhalation and triggering the release of the medicament) may vary with the relative lung function of the patient, an integrated system comprising all the above functionalities is highly preferred.

Also this feature will enhance adherence and will also enable and empower the patient to respond faster and more adequately to changes in his/her disease condition.

Further, it is preferred that the system according to the invention further comprises means for comparing a measurement of the inhalation technique with one or more predetermined estimates of sufficient or optimal inhalation technique applicable to one or more inhalation devices used for delivering a medicinal product to the animal or human subject, whereby a measurement of the relative inhalation technique is obtained.

Also this part of the system would provide beneficial information to the patient, and it is therefore highly preferred that the system further comprises means for providing a second feedback to the human or animal subject, the second feedback comprising either an approval of the inhalation technique or a suggestion of an alternative inhalation technique, the second feedback being based on a measurement of the relative inhalation technique.

Alternatively, the second suggestion may be to suggest an alternative device in case the relative inhalation technique is far from adequate.

Preferably, the system is capable of providing both the relative inhalation technique and the relative lung function. With these relative values, it is even possible for the system to suggest both a suitable inhalation device and a suitable medicament for the patient's particular state of disease.

Thus, in a highly preferred embodiment, the system comprises means for providing a third feedback to the human or animal subject, the third feedback comprising a suggestion of a suitable inhalation device and a suitable medicament, the third feedback being based on a measurement of the relative lung function and a measurement of the relative inhalation technique.

A trained doctor would be able to provide the system with adequate values at which particular devices and medicaments should be suggested by the system.

In a highly preferred aspect of the invention, the system comprises means for estimating the geographical location of the device, such as a GPS tracker. Data on geographical locations and measurements of lung function may be analysed providing an opportunity to the patient to identify geographical conditions affecting the disease. Similarly, weather conditions are preferably collected and stored automatically by the system.

Further, the determination of the geographical location may aid in the monitoring of younger and elderly patients or other patients who cannot be considered capable to administer the correct medicament in a given situation, such as in a situation of emergency.

In order to improve the ability of the patient to identify events or conditions triggering a particular disease condition, it is highly preferred that the system comprises means for assigning particular comments or observations to any particular measurement of the relative lung function. Preferably, these means are electronic means that upon any abnormal relative lung function observation will request the user to answer specific questions or provide specific informational input to the system. Such specific questions and information would preferably relate to one or more the following; weather conditions, performance of exercise, time of day, diet, calendar schedule, etc. Other information could be the recording of symptoms, rescue medication SABA etc.

Information regarding the specific abnormal relative lung function and further having assigned particular comments to each abnormal relative lung function will provide the patient with the ability to identify specific reoccurring observations that may be triggering the particular abnormal state of disease.

Thus, in a preferred aspect, the invention relates to a system as described above comprising means for comparing information entered into the system, upon two or more times of measurements of the relative lung function, to identify information appearing continuously at a particular relative lung function.

In a highly preferred aspect of the invention, the system is capable of automatically distributing the information retrieved by the system to any predefined electronic location.

Supplying information to a larger community could form the basis for improvements and reoccurring triggering events to be identified on a community basis. In addition, it is well known from several studies that social networking platforms for self-management via knowledge exchange are valuable especially when mediated by healthcare professionals.

In addition, automatically supplying predefined types of information, e.g. to a mobile phone of a caregiver (e.g. a doctor or parent or another patient), would enable the caregivers to monitor the state of disease of their patients/children.

Accordingly, in a highly preferred aspect, the invention relates to a system as described above comprising means for distributing predefined data provided by the system to pre-defined electronic locations or individuals, thereby providing a third party awareness of the condition of a human or animal subject.

Further, the system should preferably be able to distribute predefined information to several electronic locations, such as providing caregivers with acute information and providing historical data to the patient, the caregiver and/or the community or the relevant doctor.

Preferably the ability to receive information, e.g. by phone or by electronic means (such as text messages), from predefined persons should be integrated into the system, providing caregivers with an opportunity to provide relevant feedback to a patient in need thereof.

Thus, preferably the system as described above comprises means for obtaining feedback from the pre-defined electronic locations or individuals or communities.

The device and system according to the invention may thus be used to identify the best possible inhalation technique of a patient using a particular prescribed inhaler—and thereby obtain optimal inhalation that will create the optimal aerosol of medicine for that particular inhaler so the medicine reach the lungs. It is well know that patients need to train their inhalation technique at a regular basis to ensure continuous optimal inhalation. Accordingly, the device and system according to the invention may also be used to train the technique to maintain an optimal inhalation technique level. The device and system according to the invention may also be used to learn how to use new inhalers. The device and system according to the invention may also be used to train and manage different inhalation techniques, when different inhalers are used by the patient at the same time.

In a preferred aspect, the system comprises the device electronically connected to a mobile phone (a SMART phone) and an App. The system preferably comprises an attack alert function, i.e. a means of providing immediate alert to caregivers in a given order (set by the caregiver), e.g. by pressing an alert function on the app. Preferably, the caregiver will be able to provide feed-back (e.g. a to-do message that pops up on the telephone) with clear instructions to the patient on how to act.

The device and system according to the invention are preferably used to monitor the state of disease in the airways of a human or animal subject and to identify conditions and agents inducing the state of disease, and to identify the day-to-day changes in the state of the disease.

Accordingly, the invention relates to the use of the device and system described above to monitor the state of disease in the airways of a human or animal subject.

In one aspect, the device and system are used to identify the day-to-day changes in the state of the disease. Thus, the invention also relates to a method of identifying day-to-day changes in the state of the disease, the method comprising monitoring the state of the disease using a device or a system according to the invention.

In a preferred aspect, the device is used to identify conditions and/or agents inducing the state of the disease. Thus, the invention also relates to a method of identifying conditions and/or agents inducing the state of disease, the method comprising monitoring the state of the disease using a device or a system according to the invention and assigning particular conditions and/or agents reoccurring under specific conditions as conditions and agents triggering the specific condition.

The invention thus relates to a method for identifying conditions continuously triggering a particular state of disease, the method comprising the steps of:
a. providing two or more similar measurement of the subject's relative lung function,
b. assigning observations on one or more conditions, such as the location, weather conditions, altitude, social setting, level of activity, at each of the measurements of step a,
c. assigning a frequency of appearance to the one or more conditions provided in step b,
d. identifying the conditions having a high frequency of appearance at the measurements of step a as conditions triggering a particular state of disease.

Preferably, the method also includes the recording of symptoms.

In a preferred embodiment, the device or the system carries a means for tracing the geographical location (e.g. a GPS tracker) and a data storage device enabling the user to collect data at any time. Preferably the data are collected automatically in connection with disease monitoring.

Thus, according to this aspect of the invention, the invention relates to a new portable device capable of 1) measuring the lung function of the user of the device and 2) a means for tracing environmental conditions surrounding the patient (e.g. means for tracing the time and/or the geographical location (e.g. a GPS tracker)) and a data storage device enabling the user to collect data at the time of disease monitoring. Thereby, the user may monitor the state of disease and correlate the data with environmental data, e.g. in situations where the user cannot be expected to be capable of adequately monitoring the time and/or the geographical location at the time of disease monitoring.

Accordingly, the invention also relates to a device for measuring the lung function, the device comprising a first part capable of measuring the time (s) and volume of air/time (v/s) exhaled from the human subject during a single exhalation, thereby providing a measurement of the lung function, and further comprising a second part capable of monitoring the time and/or the geographical location (e.g. a GPS tracker)) and a data storage device enabling the user, preferably automatically, to collect and store data at the time of disease monitoring.

In another preferred aspect, the device and system of the invention are used to monitor the inhalation techniques of a human or animal subject suffering from a disease in the airways to improve the inhalation technique of the subject and thereby ensuring the best possible medicine deposition in the lungs for that particular inhaler Thus, the invention also relates to a method of improving the inhalation techniques of a human or animal subject, the method comprising monitoring the inhalation techniques of the subject using a device or a system according to the invention and providing feedback to the subject, the feed-back comprising a suggestion on possibilities for improving the inhalation techniques.

The invention thus relates to a method for training the inhalation techniques in the airways of a human or animal subject, the method comprising the steps of:
a. providing a measurement of the subject's relative inhalation technique using the device or system according to the invention,
b. providing a suggestion to change the inhalation speed and/or the inhaled volume to improve the subject's relative inhalation technique, the suggestion being based on one or more predetermined estimates of the sufficient or optimal inhalation technique applicable to one or more (prescription) inhalation devices used for delivering a medicinal product to the animal or human subject,
c. repeating steps a and b one or more times.

The optimal inhalation technique differs with respect to the resistance of the prescribed inhaler, medicament formulation and particle size of medicaments. The optimal technique for a given medicament and a given inhalation device or inhaler is set by the manufacturer in the form of an optimal range of acceleration and volume of inhalation.

The device and system may also be used to increase the efficacy of the medicinal products. Thereby the device and system may also be used to reduce/increase and/or optimize the intake of medicine.

1. A device for measuring the lung function and the inhalation technique of a human subject, the device comprising a first part capable of measuring the time (s) and volume of air/time (v/s) exhaled from the human subject during a single exhalation, thereby providing a measurement of the lung function, and further comprising a second part capable of measuring the acceleration (m/s²) of air and volume of inhalation from the human subject during a single inhalation, thereby providing a measurement of the inhalation technique, the second part of the device further comprising means for applying metered resistance to the inhalation, the applied resistance impairing the acceleration (m/s2) of air and volume of inhalation from the human subject during the inhalation.

2. A device according to claim 1, wherein the first part and the second part are integrated into a single unit where the entry point of exhaled air is at the same location as the exit point of inhaled air.

3. A system for monitoring the state of disease in the airways of a human or animal subject, said systems comprising:
a. a device according to claim 1,
b. a data storage device comprising means for collecting and storing one or more measurements of the lung function,
c. means for comparing a measurement of the lung function with previously obtained measurements of the lung function of the human or animal subject, thus obtaining a measurement of the relative lung function.

4. A system according to claim 3, said system comprising means d) for providing a first feedback to the human or animal subject, the first feedback being a suggestion of a suitable drug substance for treating the human or animal subject, the suggestion being based on a particular measurement of the relative lung function.

5. A system according to claim 3, wherein the system further comprises means e) for comparing a measurement of the inhalation technique with one or more predetermined estimates of a sufficient or optimal inhalation technique applicable to one or more inhalation devices used for delivering a medicinal product to the animal or human subject, thereby obtaining a measurement of the relative inhalation technique.

6. A system according to claim 5, said system comprising means f) for providing a second feedback to the human or animal subject, the second feedback comprising either a confirmation of an adequate inhalation technique or a suggestion of an alternative inhalation technique, the second feedback being based on a measurement of the relative inhalation technique.

7. A system according to claim 5, said system comprising means g) for providing a third feedback to the human or animal subject, the third feedback comprising a suggestion of a suitable inhalation device and a suitable medicament, the third feedback being based on a measurement of the relative lung function and a measurement of the relative inhalation technique.

8. A system according to claim 3, said system comprising means h) for estimating the geographical location of the device.

9. A system according to claim 3, said system comprising means i) for assigning particular comments or observations to any particular measurement of the relative lung function.

10. A system according to claim 3, said system comprising means j) for comparing data provided by means h) and i) of the system, upon two or more times of measurements of the relative lung function to identify data, comments or and observations appearing continuously at a particular relative lung function.

11. A system according to claim 3, said system comprising means k) for distributing data provided by means a-c) to pre-defined electronic locations or individuals, thereby providing a third party awareness of the condition of the human or animal subject.

12. Use of the device according to claim 1, or the system according to claim 3 for monitoring the state of disease in the airways of a human or animal subject.

13. Use of the device according to claim 1, or the system according to claim 3 for monitoring the inhalation technique of a human or animal subject suffering from a disease in the airways.

14. A method for training the inhalation techniques of the airways of a human or animal subject, the method comprising the steps of:
a. providing a measurement of the subject's relative inhalation technique,
b. providing a suggestion to change the inhalation speed and/or the inhaled volume to improve the subject's relative inhalation technique, the suggestion being based on one or more predetermined estimates of a sufficient or optimal inhalation technique applicable to one or more inhalation devices used for delivering a medicinal product to the animal or human subject,
c. repeating steps a and b one or more times.

15. A method for identifying conditions triggering a particular state of disease, the method comprising the steps of:
a. providing two or more similar measurement of the subject's relative lung function,
b. assigning observations to one or more conditions, such as the location, weather condition, altitude, social setting, level of activity, at each of the measurements of step a,
c. assigning a frequency of appearance to the one or more conditions provided in step b,
d. identifying the conditions having a high frequency of appearance at the measurements of step a, as conditions triggering the particular state of disease or relative lung function.
